(19) United States
(12) Patent Application Publication
(10) Pub. No.: US 2026/0276515 A1
(43) Pub. Date: Sep. 17, 2026

(54) TEST SYSTEM OF ROCK MASS FOR SEEPAGE COUPLING AND PERMEABILITY TESTING UNDER DEEP COMPLEX CONDITIONS

(71) Applicant: SICHUAN UNIVERSITY, Chengdu (CN)

(72) Inventors: Jianfeng LIU, Chengdu (CN); Heping XIE, Chengdu (CN); Yang WU, Chengdu (CN); Yifan TANG, Chengdu (CN); Zhenxing JI, Chengdu (CN); Fujun XUE, Chengdu (CN); Yisong DING, Chengdu (CN); Jinbing WEI, Chengdu (CN)

(73) Assignee: SICHUAN UNIVERSITY, Chengdu (CN)

(21) Appl. No.: 19/448,141

(22) Filed: Jan. 14, 2026

(30) Foreign Application Priority Data
Mar. 11, 2025 (CN) 202510285446.1

(51) Int. Cl. G01N 15/08; G01N 33/24

(52) U.S. Cl. CPC G01N 15/0826; G01N 15/0806; G01N 33/24

(57) ABSTRACT

The provided is a test system of a rock mass for seepage coupling and permeability testing under deep complex conditions. The test system includes: a seepage medium supply module, a seepage coupling module, a transient method test module, and a steady-state method test module, where the seepage medium supply module is configured to provide a seepage medium or a purging medium to the test system; the seepage coupling module is a rock mechanics testing machine; the transient method test module is configured to calculate permeability of a specimen by measuring a change in a pressure difference between an inlet and an outlet of a triaxial chamber of the rock mechanics testing machine, and is suitable for a low-permeability medium; and the steady-state method test module is configured to test a flow rate at the outlet of the triaxial chamber of the rock mechanics testing machine based on a steady-state method.

### CROSS-REFERENCE TO THE RELATED APPLICATIONS

This application is based upon and claims priority to Chinese Patent Application No. 202510285446.1, filed on Mar. 11, 2025, the entire contents of which are incorporated herein by reference.

### TECHNICAL FIELD

The present disclosure belongs to the field of rock mechanics testing for engineering rock masses, and particularly relates to a test system of a rock mass for seepage coupling and permeability testing under deep complex conditions.

### BACKGROUND

With the continuous advancement of engineering technology and the growing demands of underground construction, challenges confronting deep underground engineering have become increasingly complex. In particular, the complex coupling effects of deep underground temperature, stress, seepage, and engineering disturbance have rendered the failure mechanisms of surrounding rocks more sophisticated and variable. Deep underground engineering includes deep-buried transportation engineering, deep-buried extra-long tunnel engineering, deep-buried hydropower engineering, geological carbon sequestration in deep saline aquifers, deep geological disposal of high-level radioactive waste, deep underground energy storage facilities, deep shale gas exploitation and geothermal extraction engineering, and deep drilling engineering. These projects not only involve seepage issues, but are also subjected to the coupling effects of complex temperature fields, stress fields, and excavation-induced disturbances. These factors further accelerate the degradation of mechanical properties and the destabilization of surrounding rocks, giving rise to prominent seepage problems and engineering disasters triggered by different complex conditions. Different engineering contexts present distinct geological complexities. For example, coal-seam-crossing tunnels under complex conditions are confronted with more prominent construction technology and safety challenges. Without a thorough understanding of the surrounding rock masses of coal-seam-crossing tunnels, it is impossible to formulate effective solutions to ensure construction, which may lead to construction delays, cost overruns, or even serious safety accidents and heavy losses during tunnel construction. Therefore, developing effective testing equipment and implementing tests to characterize the seepage evolution of caprock and various complex coupling effects under different deep underground engineering conditions remain one of the urgent key problems to be solved in deep underground engineering research.

In underground rock mass engineering, the interaction between seepage media (such as gas or liquid) and the rock mass is inevitable. The seepage media can degrade the mechanical properties of the rock, while rock degradation in turn alters seepage characteristics. That is, both mechanical deterioration and changes in seepage behaviors may adversely affect engineering performance. Hence, testing and evaluating the mechanical degradation and continuous evolution of seepage in rock masses under fluid-solid coupling are essential aspects of engineering research. However, effectively conducting tests on mechanical deterioration and seepage evolution under fluid-solid coupling remains a technical challenge in rock mechanics experimentation. Although the MTS815 rock mechanics test system is an advanced apparatus in this field, it currently supports only liquid-phase seepage tests. It is incapable of performing gas-rock coupling tests and lacks a unified system for testing both liquid and gas seepage media. Moreover, during liquid seepage testing with the MTS815 rock mechanics test system, if the confining pressure fluid enters the seepage test module, it is impossible to determine whether the intrusion occurs from the top or the bottom of the rock specimen. Additionally, the module's complex structure makes it difficult to clean, allowing the residual confining fluid to remain inside. This contamination can lead to unreliable or erroneous results in subsequent tests. Furthermore, the test procedure with the MTS815 rock mechanics test system involves numerous manually operated valves. Any operational error may render the entire test futile and even damage the equipment, making the testing process operationally challenging, cumbersome, costly, time-consuming, and labor-intensive.

### SUMMARY

To overcome the shortcomings of the MTS815 rock mechanics test system, an objective of the present disclosure is to provide a test system of a rock mass for seepage coupling and permeability testing under deep complex conditions. The present disclosure realizes convenient switching between liquid and gas media and can promptly detect the location of a failure in the specimen's protective layer during testing, thereby enabling the test to be terminated in a timely manner.

The present disclosure adopts the following technical solution.

A test system of a rock mass for seepage coupling and permeability testing under deep complex conditions includes: a seepage medium supply module, a seepage coupling module, a transient method test module, and a steady-state method test module, where
the seepage medium supply module 1 is configured to provide a seepage medium or a purging medium to the test system;
the seepage coupling module is a rock mechanics testing machine; the rock mechanics testing machine includes a triaxial chamber; and the triaxial chamber includes a top end provided with a seepage medium inlet and a bottom end provided with a seepage medium outlet;
the transient method test module includes an upstream branch pipe, a first pressure sensor, a first pressure chamber, a downstream branch pipe, a second pressure sensor, a second pressure chamber, and a pressure difference sensor;
the upstream branch pipe includes an inlet end connected to a seepage medium outlet of the seepage medium supply module and an outlet end connected to the seepage medium inlet of the triaxial chamber; the first pressure chamber is connected to the upstream branch pipe through a first pressure branch pipe; and the first pressure sensor is connected to a connecting path between the upstream branch pipe and the first pressure chamber, and is configured to detect a pressure of the upstream branch pipe;
the downstream branch pipe includes an inlet end connected to the seepage medium outlet of the seepage medium supply module and an outlet end connected to the seepage medium outlet of the triaxial chamber; the second pressure chamber is connected to the downstream branch pipe through a second pressure branch pipe; and the second pressure sensor is connected to a connecting path between the downstream branch pipe and the second pressure chamber, and is configured to detect a pressure of the downstream branch pipe;
the pressure difference sensor is disposed between the upstream branch pipe and the downstream branch pipe, and is configured to detect a pressure difference between the upstream branch pipe and the downstream branch pipe; and
the steady-state method test module is connected to the downstream branch pipe, and is configured to test a fluid flow rate at the seepage medium outlet of the triaxial chamber.

Furthermore, the steady-state method test module includes a first flow meter, a second flow meter, and a first electromagnetic three-way valve; and
the first flow meter and the second flow meter are connected to a main steady-state test pipe through a branch steady-state test pipe; and the first electromagnetic three-way valve is disposed at a junction of the main steady-state test pipe and the branch steady-state test pipe, and is configured to select one of the first flow meter and the second flow meter to communicate with the main steady-state test pipe.

Furthermore, the first flow meter is a soap film flow meter (SFFM).

Furthermore, a steady-state pressure reducing valve, a first steady-state shut-off valve, and a second steady-state shut-off valve are further disposed on the main steady-state test pipe; and
the second steady-state shut-off valve and the steady-state pressure reducing valve are connected in parallel on the main steady-state test pipe and are connected in series with the first steady-state shut-off valve.

Furthermore, a heating and pressure stabilizing module is disposed between the seepage medium supply module and the transient method test module; and
the heating and pressure stabilizing module includes a first temperature sensor, a heater, a thirteenth shut-off valve, a pressure stabilizer, a second pressure reducing valve, and a second temperature sensor successively connected in series on a medium flow path.

Furthermore, a drying module and a fourteenth shut-off valve are disposed between the seepage medium supply module and the heating and pressure stabilizing module; and the drying module and the fourteenth shut-off valve are connected in parallel on the medium flow path; and
the drying module includes a dryer; a fourth shut-off valve is disposed between an inlet end of the dryer and the seepage medium outlet of the seepage medium supply module; and a fifth shut-off valve is disposed between an outlet end of the dryer and a medium inlet of the heating and pressure stabilizing module.

Furthermore, the test system further includes a vacuum module; the vacuum module includes a vacuum pump; the vacuum pump is connected to a connecting path between the dryer and the seepage medium supply module through a vacuum branch pipe; and the vacuum branch pipe is provided with a third shut-off valve for controlling the closing of the vacuum branch pipe.

Furthermore, the seepage medium supply module includes a main medium supply pipe, a compressor, a booster valve, a three-way valve, a gas source, and a check valve;
the main medium supply pipe includes one end provided with a medium inlet and another end provided with a medium outlet; and the check valve is disposed at the medium outlet of the main medium supply pipe;
the compressor communicates with the medium inlet of the main medium supply pipe through a first branch medium supply pipe; and the first branch medium supply pipe is provided with the booster valve;
the gas source communicates with the medium inlet of the main medium supply pipe through a second branch medium supply pipe; the second branch medium supply pipe is source and the medium inlet of the main medium supply pipe; and
the gas source communicates with the first branch medium supply pipe through the three-way valve; an outlet of the booster valve is connected to an inlet of the three-way valve; and the three-way valve includes one outlet connected to an inlet of the gas source and the other outlet communicating with the medium inlet of the main medium supply pipe.

Furthermore, the test system includes a data acquisition and display module, where the data acquisition and display module is connected to the first pressure sensor, the second pressure sensor, and the pressure difference sensor, and is configured to acquire and store various sensor parameters in real time.

Furthermore, the test system further includes an automatic control module; the automatic control module includes a controller; and the controller includes an input port connected to the first pressure sensor and the second pressure sensor and an output port connected to valves for controlling the opening and closing of the seepage medium supply module, the transient method test module, and the steady-state method test module.

The present disclosure has the following beneficial effects:
(1) The present disclosure enables testing of both liquid and gas seepage media, overcoming the single-function limitation of conventional test systems (i.e. one test system can only test one medium). The present disclosure realizes coupling tests of different media with rocks in the same device, solving the problem of inconsistent standards for comparative tests on the influence of different seepage media on rock mass deterioration.
(2) The present disclosure realizes continuous testing of seepage evolution from low permeability (10⁻²¹ m²) to high permeability (10⁻¹³ m²) during the whole process of the rock from intact state to the occurrence of microcracks, followed by crack propagation, and finally to the gradual deterioration of the rock mass.
(3) The present disclosure offers simple test operation, and prevents damage to the equipment or the specimen during testing. Multiple tests can be performed for the same test state, which not only reduces the test cost but also ensures the reliability of the test results.
(4) The present disclosure realizes mutual feedback tests between mechanical properties and seepage behaviors of different seepage media under the coupling of various construction and operation modes (i.e. different excavation and mining methods) during engineering construction and operation. The present disclosure reflects the influence of the in-situ deep underground environment where the rock mass is located, overcoming the limitation of conventional test systems to single engineering construction states and single seepage coupling media.

### BRIEF DESCRIPTION OF THE DRAWINGS

FIG. 1 is a schematic diagram of a test system of a rock mass for seepage coupling and permeability testing under deep complex conditions according to the present disclosure;

FIG. 2 shows a photo of a gypsum rock specimen in a test example;

FIG. 3 is a distribution diagram of seepage measuring points of the gypsum rock specimen during a permeability test in the test example;

FIGS. 4A-4D show a stress-strain curve of the gypsum rock specimen under four different confining pressure seepage conditions at room temperature in the test example;

FIG. 5 shows a variation in permeability of the gypsum rock specimen in each confining pressure group throughout a loading process in the test example; and

FIGS. 6A-6D show a characteristic curve of a variation in permeability of the gypsum rock with a loading stress under different confining pressures at room temperature in the test example.
Reference Numerals: 1. seepage medium supply module; 11. compressor; 12. booster valve; 13. three-way valve; 14. gas source; 15. check valve; 16. first shut-off valve; 17. second shut-off valve; and 18. first pressure reducing valve;
2. vacuum module; 21. vacuum pump; 22. vacuum branch pipe; and 23. third shut-off valve;
3. drying module; 31. dryer; 32. fourth shut-off valve; and 33. fifth shut-off valve;
4. data acquisition and display module;
5. transient method test module; 51. upstream branch pipe; 52. first pressure sensor; 53. first pressure chamber; 54. downstream branch pipe; 55. second pressure sensor; 56. second pressure chamber; 57. pressure difference sensor; 58. sixth shut-off valve; 59. seventh shut-off valve; 510. eighth shut-off valve; 511. ninth shut-off valve; 512. tenth shut-off valve; 513. eleventh shut-off valve; 514. twelfth shut-off valve; 515. first safety valve; and 516. second safety valve;
6. heating and pressure stabilizing module; 61. first temperature sensor; 62. heater; 63. pressure stabilizer; 64. second pressure reducing valve; 65. second temperature sensor; and 66. thirteenth shut-off valve;
7. steady-state method test module; 71. first flow meter; 72. second flow meter; 73. first electromagnetic three-way valve; 74. steady-state pressure reducing valve; 75. first steady-state shut-off valve; 76. second steady-state shut-off valve; and 77. second electromagnetic three-way valve;
8. seepage coupling module; 81. triaxial chamber; and 82. specimen; and
9. fourteenth shut-off valve.

### DETAILED DESCRIPTION OF THE EMBODIMENTS

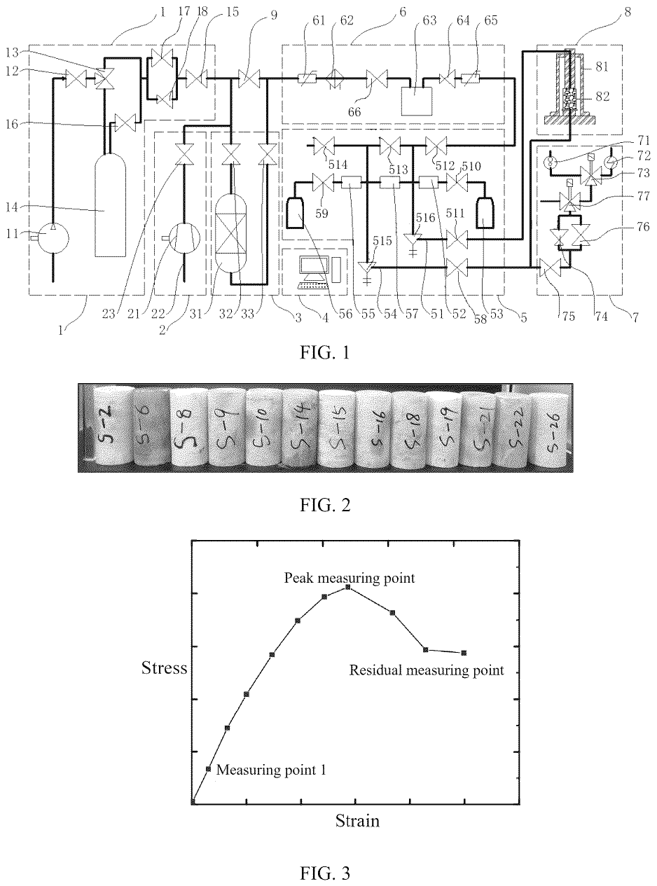

The present disclosure is further described below with reference to FIGS. 1 to 6, an embodiment, and a test example.

### Embodiment

The present disclosure provides a test system of a rock mass for seepage coupling and permeability testing under deep complex conditions. As shown in FIG. 1, the test system includes seepage medium supply module 1, seepage coupling module 8, transient method test module 5, and steady-state method test module 7. The seepage medium supply module 1 is configured to provide a seepage medium or a purging medium with a stable pressure to the test system.

Specifically, the seepage medium supply module 1 is configured to provide a seepage medium or a purging medium to the test system. The seepage medium supply module 1 includes a main medium supply pipe, compressor 11, booster valve 12, three-way valve 13, gas source 14, and check valve 15. The main medium supply pipe includes one end provided with a medium inlet and another end provided with a medium outlet. The compressor 11 communicates with the medium inlet of the main medium supply pipe through a first branch medium supply pipe. The check valve 15 is disposed at the medium outlet of the main medium supply pipe. The booster valve 12 is disposed between the compressor 11 and the three-way valve 13.

To realize the diversification of gas supply modes, the gas source 14 is disposed on a second branch medium supply pipe. An inlet of the gas source 14 is connected to three-way valve 13, and an outlet of the gas source 14 is connected to the check valve 15 through first shut-off valve 16.

An inlet end of the check valve 15 is provided with second shut-off valve 17 and first pressure reducing valve 18 that can change a gas supply mode. The second shut-off valve 17 and the first pressure reducing valve 18 are connected in parallel and are connected to the check valve 15.

In this way, the seepage medium supply module 1 can supply a gas independently through the compressor 11, independently through the gas source 14, or first supply the gas to the gas source 14 through the compressor 11 and then supply the gas to the test system. The gas supply mode is selected according to actual conditions, and each gas supply mode can form a backup for each other.

The seepage coupling module 8 is a rock mechanics testing machine, which includes a triaxial chamber. The triaxial chamber includes a top end provided with a seepage medium inlet and a bottom end provided with a seepage medium outlet. The triaxial chamber is selected because compared with uniaxial compression tests, triaxial compression tests consider the effect of confining pressure, which is more in line with the stress state of surrounding rocks in underground engineering and can more truly reflect the real mechanical properties of surrounding rocks in underground engineering.

The transient method test module 5 includes upstream branch pipe 51, first pressure sensor 52, first pressure chamber 53, downstream branch pipe 54, second pressure sensor 55, second pressure chamber 56, and pressure difference sensor 57.

Specifically, the upstream branch pipe 51 includes an inlet end connected to the seepage medium outlet of the seepage medium supply module 1 to form a medium flow path and an outlet end connected to the seepage medium inlet of the triaxial chamber 81. The first pressure chamber 53 is connected to the upstream branch pipe 51 through the first pressure branch pipe. The first pressure sensor 52 is connected to a connecting path between the upstream branch pipe 51 and the first pressure chamber 53 to detect a pressure of the upstream branch pipe 51. The downstream branch pipe 54 includes an inlet end connected to the seepage medium outlet of the seepage medium supply module 1 to form the medium flow path and an outlet end connected to the seepage medium outlet of the triaxial chamber 81. The second pressure chamber 56 is connected to the downstream branch pipe 54 through a second pressure branch pipe. the second pressure sensor 55 is connected to a connecting path between the downstream branch pipe 54 and the second pressure chamber 56 to detect a pressure of the downstream branch pipe 54. The pressure difference sensor 57 is connected between the upstream branch pipe 51 and the downstream branch pipe 54 to detect a pressure difference between the upstream branch pipe 51 and the downstream branch pipe 54.

It should be noted that to ensure the equivalent bulk of the upstream branch pipe 51 and the downstream branch pipe 54 and reduce the floor space, the first pressure chamber 53 and the second pressure chamber 56 in this module are arranged symmetrically, the first pressure sensor 52 and the second pressure sensor 55 are arranged symmetrically, and the upstream branch pipe 51 and the downstream branch pipe 54 are arranged symmetrically.

Specifically, the maximum range of both the first pressure sensor 52 and the second pressure sensor 55 is 10 MPa. The maximum range of the pressure difference sensor 57 is 700 kPa, with an accuracy of 0.001 kPa. The first pressure chamber 53 and the second pressure chamber 56 are special high-pressure steel cylinders with a bulk of 0.5 L and a maximum bearable pressure of 40 MPa. It is necessary to prevent pressure oil of the triaxial chamber from entering a gas seepage test system and causing the pressure in the pipe to exceed the safe pressure of the pressure sensors and pressure difference sensor, thereby avoiding damage to the test system. To this end, second safety valve 516 is disposed on the upstream branch pipe 51, and first safety valve 515 is disposed on the downstream branch pipe 54.

In addition, to better control the medium flow in the upstream branch pipe 51 and the downstream branch pipe 54, tenth shut-off valve 512 is disposed at an inlet end of the upstream branch pipe. Ninth shut-off valve 511 and sixth shut-off valve 58 are arranged at an outlet end of the upstream branch pipe 51 and an inlet end of the downstream branch pipe, respectively. Eighth shut-off valve 510 and seventh shut-off valve 59 are arranged on pipes where the first pressure chamber 53 and the second pressure chamber 56 are located, respectively. Twelfth shut-off valve 514 is disposed at a free end of the downstream branch pipe 54.

It should be noted that the transient method test module 5 is designed based on a transient method. It calculates permeability of the specimen by measuring a change in a pressure difference between an inlet and an outlet of the specimen 82, and is suitable for low-permeability media.

To improve the measurement accuracy and application range, the test system of the present disclosure is further provided with the steady-state method test module 7. The steady-state method test module 7 is connected to the downstream branch pipe 54 and configured to test a fluid flow rate at a bottom outlet of the triaxial chamber 81.

Specifically, the steady-state method test module 7 includes first flow meter 71, second flow meter 72, and first electromagnetic three-way valve 73. The first flow meter 71 and the second flow meter 72 are connected to a main steady-state test pipe through a branch steady-state test pipe. The first electromagnetic three-way valve 73 is disposed at a junction of the main steady-state test pipe and the branch steady-state test pipe to select one of the first flow meter 71 and the second flow meter 72 to communicate with the main steady-state test pipe. For seepage with a small flow rate and short time, the first flow meter 71 with high sensitivity is selected. The first flow meter 71 is a soap film flow meter (SFFM). For seepage with a large flow rate and long time, the second flow meter 72 is selected. The second flow meter 72 is a conventional flow meter. In addition, the main steady-state test pipe is further provided with steady-state pressure reducing valve 74, first steady-state shut-off valve 75, and second steady-state shut-off valve 76. The second steady-state shut-off valve 76 and the steady-state pressure reducing valve 74 are connected in parallel on the main steady-state test pipe and are connected in series with the first steady-state shut-off valve 75. To connect equipment for detecting other properties of the specimen, second electromagnetic three-way valve 77 is further disposed between the first electromagnetic three-way valve 73 and the main steady-state test pipe. The second electromagnetic three-way valve 77 serves as an expansion end.

It should be noted that the steady-state method test module 7 is designed based on a steady-state method. It can test the flow rate of the fluid medium at the outlet end of the specimen 82 and is suitable for high-permeability media.

The present disclosure integrates the transient method test module and the steady-state method test module into one test system, which can measure the seepage flow rate of both low-permeability media and high-permeability media, and has a wide application range.

To increase the temperature of the seepage medium and ensure the stable pressure of the test system, heating and pressure stabilizing module 6 is disposed between the seepage medium supply module 1 and the transient method test module 5. The heating and pressure stabilizing module 6 includes first temperature sensor 61, heater 62, thirteenth shut-off valve 66, pressure stabilizer 63, second pressure reducing valve 64, and second temperature sensor 65 successively connected in series on the medium flow path.

The advantage of this design is that the seepage medium is heated by the heater 62 to effectively increase the temperature of the seepage medium. Thus, when the gas pressure is insufficient, a stable gas pressure can be provided by increasing the temperature of the seepage medium. Moreover, it is easy to operate and has a simple structure.

To dry the seepage medium and system pipes and improve the test accuracy, drying module 3 and fourteenth shut-off valve 9 are disposed between the seepage medium supply module 1 and the heating and pressure stabilizing module 6. The drying module 3 and the fourteenth shut-off valve 9 are connected in parallel on the medium flow path.

Specifically, the drying module 3 includes dryer 31. Fourth shut-off valve 32 is disposed between an inlet end of the dryer 31 and the seepage medium outlet of the seepage medium supply module 1, and fifth shut-off valve 33 is disposed between an outlet end of the dryer 31 and a medium inlet of the heating and pressure stabilizing module 6.

To detect the tightness of the entire test system, vacuum module 2 is further provided. The vacuum module 2 includes vacuum pump 21. The vacuum pump 21 is connected to a connecting path between the dryer 31 and the seepage medium supply module 1 through vacuum branch pipe 22. Third shut-off valve 23 for controlling the closing of the vacuum branch pipe 22 is disposed on the vacuum branch pipe 22.

Furthermore, the test system further includes data acquisition and display module 4. The data acquisition and display module 4 is connected to the first pressure sensor 52, the second pressure sensor 55, and the pressure difference sensor 57, and is configured to acquire and store various sensor parameters in real time.

The data acquisition and display module 4 mainly includes a data acquisition card, a computer system, and a software system. The working principle of the data acquisition and display module belongs to prior art, so it will not be repeated herein.

To realize automatic control to avoid manual opening of various valves and improve test accuracy, the test system further includes an automatic control module (not shown in the figure). The automatic control module includes a controller. The controller includes an input port connected to the first pressure sensor 52 and the second pressure sensor 55 and an output port connected to the valves that control the opening and closing of the seepage medium supply module 1, the transient method test module 5, and the steady-state method test module 7. In addition, the automatic control module makes the test system easy to operate.

The test system is suitable for both gas media and liquid media, realizes the coupling test of different media with rocks in the same device, and solves the problem of inconsistent standards for comparative tests on the influence of different seepage media on rock mass deterioration. When the test system is applied to a liquid medium, the liquid enters from the main supply pipe through the compressor 11, and only the gas source 14 and the drying module 3 are closed at this time.

### Test Example

In this test example, the test system of a rock mass for seepage coupling and permeability testing under deep complex conditions in the embodiment is used to perform a gas-solid coupling and seepage test on a gypsum rock specimen taken from a caprock of an underground gas storage, so as to reveal the evolution law of the seepage mechanical behavior of the gypsum rock under gas-solid coupling. The seepage medium during the test is nitrogen.

### (1) Specimen Preparation

The gypsum rock is precision processed and polished indoors in accordance with the “Standard for Test Methods of Engineering Rock Masses” and “Code for Rock Tests of Hydroelectric and Water Conservancy Engineering” to prepare a standard specimen for an indoor rock mechanics test. The specimen has a size of Φ×H=38×76 mm (diameter×height), and artificial seepage holes with a diameter of 3 mm are provided at two ends of the specimen, as shown in FIG. 2. The specimen is stored dry before the test.

### (2) Experimental Equipment

An MTS815 rock mechanics test system is used.

### (3) Test Method

(1) The gypsum rock specimen wrapped with a heat-shrinkable film is disposed in the triaxial chamber.
(2) Confining pressure is applied to the gypsum rock specimen. The confining pressure comes from the pressure oil in the triaxial chamber, and the pressure oil surrounds the gypsum rock specimen. The confining pressure is applied at 3 MPa/min. After the confining pressure reaches a target value, the seepage medium supply module 1 is started to apply gas pressure to the two ends of the gypsum rock specimen. The gas pressure value set to 1.5 MPa. After the gas pressure is stabilized for 30 min, a first seepage test is performed, and the permeability at this point is regarded as permeability before axial loading, that is, this point is an initial point.
(3) After the permeability test at the initial point is completed, axial loading is performed. The pressure comes from a triaxial test shaft, and loading is performed until the specimen is deformed and damaged. The axial loading rate is controlled by a linear variable differential transformer (LVDT) to 0.08 mm/min.
(4) The seepage measuring points are evenly distributed throughout the loading process. When a specified stress point is reached and the load stabilizes through the LVDT, the seepage parameter at this stress point is tested using a transient method.

During this process, it is necessary to ensure that there are more than 10 measuring points during the whole process of loading and damaging the gypsum rock specimen, including 1 seepage measuring point at the peak stress point and more than 3 seepage measuring points after the peak stress. The distribution diagram of the seepage measuring points is shown in FIG. 3.
(5) After the above steps, the mechanical behavior data and seepage data are acquired for data processing.

It should be noted that there are 4 target confining pressure values measured in the step (2), which are 5 MPa, 15 MPa, 25 MPa, and 35 MPa. That is, the above test is performed 4 times, and the test method is the same each time, with the only difference being the target confining pressure, so as to more accurately acquire the mechanical behavior characteristics and seepage data of the gypsum rock specimen.

The permeability K_{P} in the transient method is calculated as follows:

$$Δ P ( t ) = Δ {P}_{0} {e}^{ω t} ;$$

$$ω = {K}_{P} \frac{A}{μ η L} ( \frac{1}{{V}_{1}} + \frac{1}{{V}_{2}} ) ;$$

where, ΔP(t) denotes a pressure difference at the two ends of the specimen at time t, unit: MPa; ΔP₀ denotes an initial pressure difference between the two ends of the specimen, unit: MPa; t denotes a duration of one seepage, unit: s; denotes a dynamic viscosity coefficient of the seepage medium nitrogen, unit: Pa·s; η denotes a compressibility factor of nitrogen, unit: Pa⁻¹; V₁ and V₂ respectively denote volumes of buffer gas cylinders at upper and lower gas pressure ends, unit: m³; A denotes a cross-sectional area of the specimen, unit: m²; and L denotes a height of the specimen, unit: m.

### (4) Test Results and Analysis

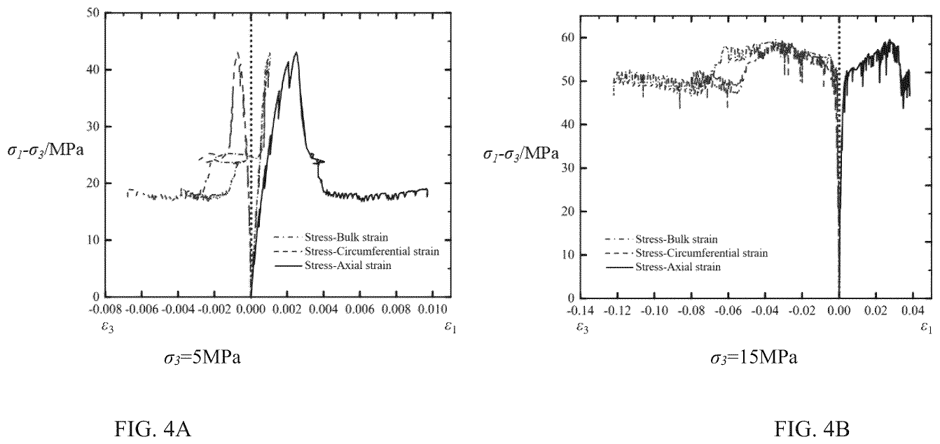

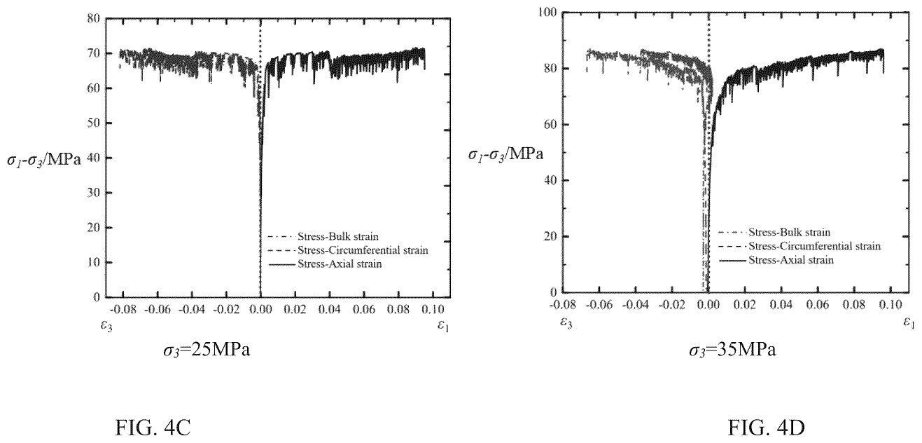

FIGS. 4A-4D show stress-strain curves of the gypsum rock specimen under four different confining pressure seepage conditions at room temperature. It can be seen from the figure that the brittle failure and ductile failure characteristics of the gypsum rock are obvious.

Under seepage conditions, the deformation stages of the gypsum rock specimen after axial loading sequentially include a crack compaction stage, a crack initiation and propagation stage, a stress oscillation stage, and a macro-crack penetration failure stage, and a strain hardening stage. Under different confining pressures, the strength and deformation characteristics of the gypsum rock specimen are shown in Table 1:

TABLE 1
Strength and deformation characteristic parameters of
gypsum rock under conventional loading-seepage loading

| | | | | | Peak |
|---|---|---|---|---|---|
| Confining | | Peak | Residual | | circum- |
| pressure/ | | deviatoric | stress/ | Peak axial | ferential |
| MPa | State | stress/MPa | MPa | strain/% | strain/% |
| 5 | Conventional | 42.94 | 33.1 | 0.26 | −0.12 |
| | Seepage | 43.55 | 19.15 | 0.25 | −0.075 |
| 15 | Conventional | 65.61 | 53.1 | 0.31 | −4.15 |
| | Seepage | 59.6 | 51.1 | 2.73 | −3.08 |
| 25 | Conventional | 78.48 | 77.78 | 8.24 | −8.5 |
| | Seepage | 72.78 | 70.98 | 8.98 | −7.72 |
| 35 | Conventional | 86.3 | 84.78 | 10.38 | −10.49 |
| | Seepage | 87.06 | 85.52 | 9.48 | −6.37 |

Note:
Conventional loading means no gas pressure is applied in the step (2) of the test method; axial strain and circumferential strain are measured by an axial extensometer and a circumferential extensometer, respectively; and stress is measured by a force sensor.

It can be seen from Table 1 that there is no significant difference in the peak deviatoric stress between the conventional loading state and the seepage condition, and the gas pressure has a weakening effect on the residual strength of gypsum rock.

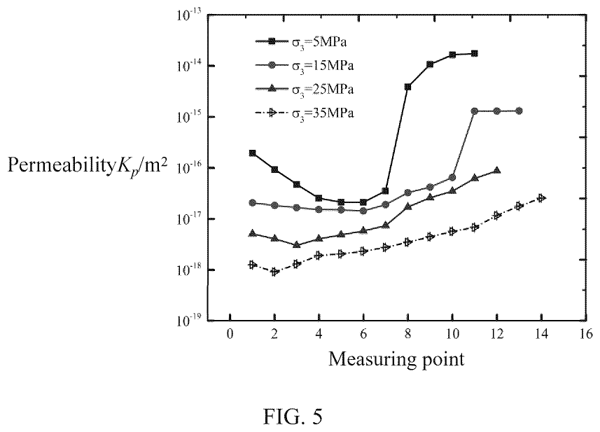

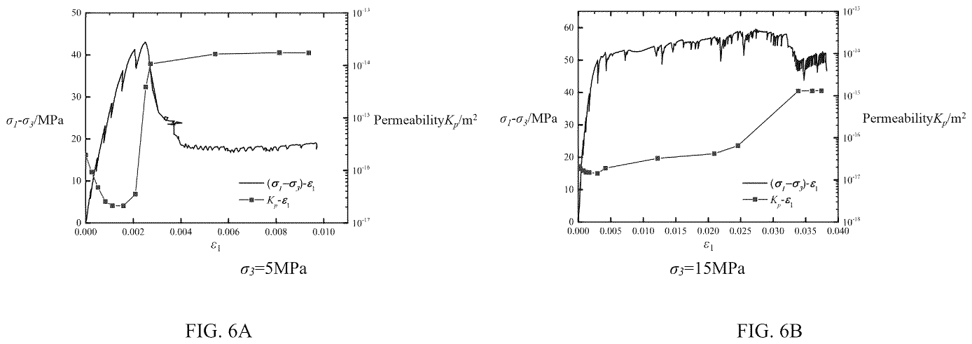

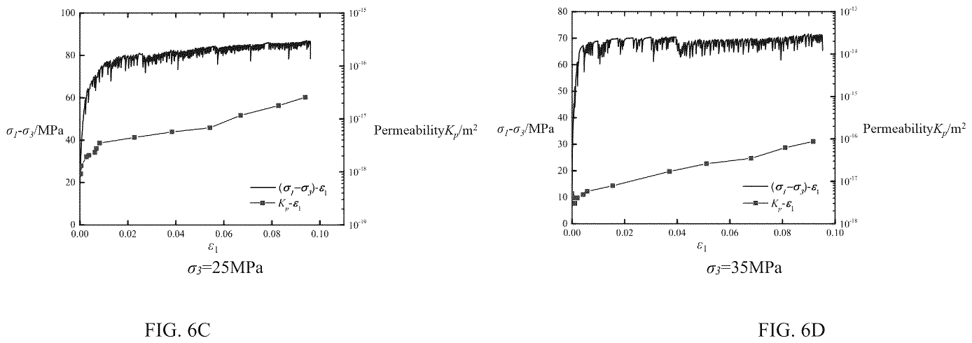

FIG. 5 shows a variation in permeability of the gypsum rock specimen in each confining pressure group throughout a loading process. It can be seen from the figure that the confining pressure inhibits the permeability. At the macroscopic level, the permeability curves show different trends under different confining pressures, but at the overall permeability level, the overall permeability level decreases with the increase of confining pressure.

FIGS. 6A-6D show a characteristic curve of a variation in permeability of the gypsum rock with a loading stress under different confining pressures at room temperature. It can be seen from the figure that the permeability mainly shows a change trend of first decreasing, then increasing, and finally tending to be stable or continuing to increase.

In summary, the present disclosure addresses the testing challenge of fluid-solid coupling mechanical mutual feedback under different mining methods in simulating deep underground coal mining. The present disclosure also addresses the testing challenge of the mechanical deterioration process of surrounding rocks in simulating the operation of deep underground energy storage projects.

Those of ordinary skill in the art will understand that the embodiments described herein are intended to help readers understand the principles of the present disclosure, and it should be understood that the protection scope of the present disclosure is not limited to such special statements and embodiments. Those of ordinary skill in the art may make other various specific modifications and combinations according to the technical teachings disclosed in the present disclosure without departing from the essence of the present disclosure, and such modifications and combinations still fall within the protection scope of the present disclosure.

What is claimed is:

1. A test system of a rock mass for seepage coupling and permeability testing under deep complex conditions, comprising: a seepage medium supply module, a seepage coupling module, a transient method test module, and a steady-state method test module, wherein
the seepage medium supply module is configured to provide a seepage medium to the test system, and the seepage medium is a liquid or a gas;
the seepage coupling module is a rock mechanics testing machine; the rock mechanics testing machine comprises a triaxial chamber; and the triaxial chamber comprises a top end provided with a seepage medium inlet and a bottom end provided with a seepage medium outlet;
the transient method test module comprises an upstream branch pipe, a first pressure sensor, a first pressure chamber, a downstream branch pipe, a second pressure sensor, a second pressure chamber, and a pressure difference sensor;
the upstream branch pipe comprises an inlet end connected to a seepage medium outlet of the seepage medium supply module and an outlet end connected to the seepage medium inlet of the triaxial chamber; the first pressure chamber is connected to the upstream branch pipe through a first pressure branch pipe; and the first pressure sensor is connected to a connecting path between the upstream branch pipe and the first pressure chamber, and is configured to detect a pressure of the upstream branch pipe;
the downstream branch pipe comprises an inlet end connected to the seepage medium outlet of the seepage medium supply module and an outlet end connected to the seepage medium outlet of the triaxial chamber; the second pressure chamber is connected to the downstream branch pipe through a second pressure branch pipe; and the second pressure sensor is connected to a connecting path between the downstream branch pipe and the second pressure chamber, and is configured to detect a pressure of the downstream branch pipe;
the pressure difference sensor is disposed between the upstream branch pipe and the downstream branch pipe, and is configured to detect a pressure difference between the upstream branch pipe and the downstream branch pipe;
a heating and pressure stabilizing module is disposed between the seepage medium supply module and the transient method test module; and the heating and pressure stabilizing module comprises a first temperature sensor, a heater, a thirteenth shut-off valve, a pressure stabilizer, a second pressure reducing valve, and a second temperature sensor successively connected in series on a medium flow path;
a drying module and a fourteenth shut-off valve are disposed between the seepage medium supply module and the heating and pressure stabilizing module; the drying module and the fourteenth shut-off valve are connected in parallel on the medium flow path; the drying module comprises a dryer; a fourth shut-off valve is disposed between an inlet end of the dryer and the seepage medium outlet of the seepage medium supply module; and a fifth shut-off valve is disposed between an outlet end of the dryer and a medium inlet of the heating and pressure stabilizing module; and
the steady-state method test module is connected to the downstream branch pipe, and is configured to test a fluid flow rate at the seepage medium outlet of the triaxial chamber.

2. The test system of the rock mass for the seepage coupling and the permeability testing under the deep complex conditions according to claim 1, wherein the steady-state method test module comprises a first flow meter, a second flow meter, and a first electromagnetic three-way valve; and
the first flow meter and the second flow meter are connected to a main steady-state test pipe through a branch steady-state test pipe; and the first electromagnetic three-way valve is disposed at a junction of the main steady-state test pipe and the branch steady-state test pipe, and is configured to select one of the first flow meter and the second flow meter to communicate with the main steady-state test pipe.

3. The test system of the rock mass for the seepage coupling and the permeability testing under the deep complex conditions according to claim 2, wherein the first flow meter is a soap film flow meter (SFFM).

4. The test system of the rock mass for the seepage coupling and the permeability testing under the deep complex conditions according to claim 3, wherein a steady-state pressure reducing valve, a first steady-state shut-off valve, and a second steady-state shut-off valve are further disposed on the main steady-state test pipe; and
the second steady-state shut-off valve and the steady-state pressure reducing valve are connected in parallel on the main steady-state test pipe and are connected in series with the first steady-state shut-off valve.

5. The test system of the rock mass for the seepage coupling and the permeability testing under the deep complex conditions according to claim 1, further comprising a vacuum module, wherein the vacuum module comprises a vacuum pump; the vacuum pump is connected to a connecting path between the dryer and the seepage medium supply module through a vacuum branch pipe; and the vacuum branch pipe is provided with a third shut-off valve for controlling closing of the vacuum branch pipe.

6. The test system of the rock mass for the seepage coupling and the permeability testing under the deep complex conditions according to claim 1, wherein the seepage medium supply module comprises a main medium supply pipe, a compressor, a booster valve, a three-way valve, a gas source, and a check valve;
the main medium supply pipe comprises one end provided with a medium inlet and another end provided with a medium outlet; and the check valve is disposed at the medium outlet of the main medium supply pipe;
the compressor communicates with the medium inlet of the main medium supply pipe through a first branch medium supply pipe; and the first branch medium supply pipe is provided with the booster valve;
the gas source communicates with the medium inlet of the main medium supply pipe through a second branch medium supply pipe; the second branch medium supply pipe is provided with a first shut-off valve; and the first shut-off valve is located between the gas source and the medium inlet of the main medium supply pipe; and
the gas source communicates with the first branch medium supply pipe through the three-way valve; an outlet of the booster valve is connected to an inlet of the three-way valve; and the three-way valve comprises one outlet connected to an inlet of the gas source and the other outlet communicating with the medium inlet of the main medium supply pipe.

7. The test system of the rock mass for the seepage coupling and the permeability testing under the deep complex conditions according to claim 1, comprising a data acquisition and display module, wherein the data acquisition and display module is connected to the first pressure sensor, the second pressure sensor, and the pressure difference sensor, and is configured to acquire and store various sensor parameters in real time.

8. The test system of the rock mass for the seepage coupling and the permeability testing under the deep complex conditions according to claim 1, further comprising an automatic control module, wherein the automatic control module comprises a controller; and the controller comprises an input port connected to the first pressure sensor and the second pressure sensor and an output port connected to valves for controlling opening and closing of the seepage medium supply module, the transient method test module, and the steady-state method test module.
